(19) United States
(12) Patent Application Publication
(10) Pub. No.: US 2010/0324607 A1
(43) Pub. Date: Dec. 23, 2010

(54) PEDICLE SCREW WITH EXPANSION ANCHOR SLEEVE

(76) Inventor: Reginald J. Davis, Cockeysville, MD (US)

Correspondence Address: OBER / KALER;C/O ROYAL W. CRAIG, 120 EAST BALTIMORE STREET, SUITE 800, BALTIMORE, MD, 21202, US

(21) Appl. No.: 12/821,945

(22) Filed: Jun. 23, 2010

Related U.S. Application Data
(60) Provisional application No. 61/269,274, filed on Jun. 23, 2009.

(51) Int. Cl. A61B 17/86

(52) U.S. Cl. 606/313

(57) ABSTRACT

A bone anchor for use in conjunction with orthopedic spinal fixation comprising a pedicle screw having a threaded proximal tip for threaded insertion into a hole prepared into a vertebral body through the pedicle of the vertebra from the posterior approach. The shank of the anchor is of smaller diameter than the proximal tip so as to receive a sleeve after insertion into the bone. The tubular sleeve is provided with a plurality of longitudinal slots evenly arranged about the longitudinal axis of the sleeve to forming a plurality of longitudinal. A tubular compression collar for insertion over said shank portion is provided on the shank and driven toward the proximal tip compression the sleeve and causing the ribs to expand radially into a star pattern securing the anchor in the bone. A tubular collar of natural or synthetic bone growth promoting material may be inserted to promote bone growth, close the hole and further stabilize the anchor. An elastomeric collar may also be inserted over the shank and extend beyond the end of the shank to flexibly receive a rod seat for receiving an orthopedic rod affixed to said elastomeric collar.

### CROSS-REFERENCE TO RELATED APPLICATIONS

This application claims the benefit of U.S. Provisional Application No. 61/269,274 for “PEDICLE SCREW WITH EXPANSION ANCHOR SLEEVE,” filed Jun. 23, 2009, which is incorporated by reference.

### BACKGROUND OF THE INVENTION

1. Field of the Invention

The present invention relates to spinal fixation devices and, more particularly to a modular screw/sleeve assembly for insertion into spinal bone for fixation of the vertebra such as by affixing orthopedic implantation structures to the spine.

2. Description of the Background

The spinal column is a highly complex arrangement of 24 individual vertebral bones separated by cartilaginous intervertebral discs supported by muscle and ligament that houses and protects critical elements of the nervous system and supports the body. Despite this role the spine is a highly flexible structure that is capable of significant articulation through a wide range of motion. In a healthy spine this flexibility is a result of its composite nature which permits relative movement between adjacent vertebra at the intervertebral discs and at the facet joints. The spinal vertebra vary in form along the length of the spine but are generally comprised of a roughly cylindrical vertebral body anterior to a central canal that houses the spinal cord. The central canal is formed and the spinal cord protected from the posterior approach by the vertebral arch, which is itself comprised of two pedicles extending laterally from the vertebral body and joined medially by the lamina. The transverse process extend laterally from the pedicle/laminar joint and the spinous process extends posteriorily and generally down from medial line where the plates of the lamina join. The cylindrical vertebral bodies are stacked and separated from each other by resilient intervertebral discs.

Genetic or developmental irregularities, trauma, disease or other causes can result in spinal pathologies which degrade the spinal structures limiting the range of motion, endangering the neural elements housed within the spinal column and causing tremendous pain to the individual. In mild cases pain and degradation of the spine can be improved or eliminated by combinations of physical and pharmaceutical therapy along with rest. In many cases surgical intervention is required after non-invasive methods have been ineffective. Surgical techniques relieve the pressures on neural structures through procedures such as laminectomy. Surgeons may fuse adjacent vertebra by full or partial discectomy and implantation of graft material and an intervertebral prosthetic device to immobilize vertebra relative to one another and reduce pain.

Numerous fusion and implant techniques have been utilized via anterior, posterior or lateral approaches to the spine. Supplementary orthopedic implants including rods and plates to support and stabilize the spine during and after the healing process are commonly employed with such fusion techniques. Lateral and anterior supplementary orthopedic assemblies are coupled to the vertebral bodies and are generally supported by screws which enter into the vertebral bodies directly. Posterior assemblies are applied to the back of the spinal column and generally utilize hooks or screws under the lamina and entering into the central canal, attaching to the transverse process, or coupling through the pedicle bone. In the thoracic and cervical spine, posterior implantation assemblies are usually supported by screws which enter into the pedicle and extend into the vertebral body from the rear.

A common mode of failure and a primary concern of surgeons performing spinal implantation is screw pull-out in which the surface of the bone in the whole that is engaged by the screw threads fails. Screw pull-out is often due to the high stress imposed on the assembly by the relative motion of the spine in everyday use but can be exacerbated by pathological weakening of the osseous tissue. Pull-out failures may result in subsequent surgical procedures to re-attach the assembly, a process that is made more difficult by the enlarged hole left by the pulled-out screw and weakened or damaged vertebral bone that is incapable of supporting the orthopedic implant.

This mode of failure is partially a function of the structure of the vertebral bone itself which is generally comprised of a relatively thin outer layer or shell of hard and strong corticle bone material around an inner center of generally softer cancellous bone. Because the corticle shell is thin and the hole into which a screw is inserted is generally perpendicular to its surface, the majority of the length of the screw is within the softer cancellous bone and relies on the engagement of the screw threads with this softer material for its strength and holding power. This is particularly true for screws that have a constant, or tapered, diameter.

There are a variety of expansive and sleeve designs for bone screws that are intended to reduce the risk of pull-out failure. Examples include United States Patent Applications 20060095040 (Schlienger et al.) and 20070038219 (Matthis) as well as U.S. Pat. Nos. 2,381,050 (Hardinge), 5,084,050 (Draenert), 5,713,904 (Errico et al.), 4,716,893 (Fischer et al.) and 7,074,203 (Johanson et al.), all using sleeves having barbs, wedges or sidewalls that expand radially or laterally on insertion of the screw. U.S. Pat. No. 6,168,597 (Biedermann et al.) expands on withdrawal of a rod. However, each of these designs continues to rely on engagement with the sidewall of the screw hole for its holding power and is thus similarly prone to failure. Moreover, the prior art designs are concerned primarily with fixation and do not address the need to add elements onto the fixation device such as bone grafts and/or medicament-leaching graft promoters.

It would be advantageous to provide a bone fixation screw assembly that relies on engagement with the hard external cortical bone of the vertebra rather than the relatively soft cancellous interior bone, and which facilitates introduction of ancillary elements such as bone grafts and/or medicament-leaching graft promoters.

### SUMMARY OF THE INVENTION

It is therefore an object of the present invention to provide a bone anchor that is resistant to pull out failure.

It is another object to provide a bone anchor that can be utilized in conjunction with bone that has previously been damaged by pullout failure of a bone screw.

It is another object to provide a bone anchor that does not rely on the engagement of the threads of a screw with the exposed bone surfaces of a hole drilled in the vertebra of a patient for its strength and ability to remain fixed in place.

It is another object to provide a bone anchor that allows insertion and facilitates introduction of ancillary elements such as bone grafts and/or medicament-leaching graft promoters.

In accordance with the foregoing objects, the present invention is a bone anchor for use in conjunction with orthopedic spinal fixation comprising an elongate screw anchor having a shank extending to a threaded proximal tip for threaded insertion into a hole prepared into a vertebral body through the pedicle of the vertebra from the posterior approach. The shank of the anchor is of smaller diameter than the proximal tip so as to receive a sleeve after insertion into the bone. The tubular sleeve is provided with a plurality of longitudinal slots evenly arranged about the longitudinal axis of the sleeve. A tubular compression collar is provided for insertion over said shank portion. When driven toward the proximal tip compression of the sleeve causes the ribs to expand radially into a star pattern securing the anchor in the bone. The bone anchor also allows insertion and facilitates introduction of ancillary elements such as bone grafts and/or medicament-leaching graft promoters. For example, a tubular collar of natural or synthetic bone growth promoting material may be inserted onto the shank of the anchor to promote bone growth, close the hole and further stabilize the anchor. An elastomeric collar may also be inserted over the shank of the anchor and extend beyond the end of the shank to flexibly receive a rod seat for receiving an orthopedic rod affixed to said elastomeric collar.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 is an exploded perspective view of an embodiment of the present invention.

FIG. 2 is a semi-transparent view of the basic screw inserted into the vertebral body via the pedicle.

FIG. 3 is a semi-transparent view of the basic screw inserted into the vertebral body with the expansion sleeve and compression collar inserted over the shank of the screw.

FIG. 4 is a semi-transparent view of the basic screw inserted into the vertebral body after the sleeve has been compressed by action of the compression collar and the folding star thus expanded.

FIG. 5 is a semi-transparent view of the distal elements of the invention positioned on the distal end of the basic screw.

### DETAILED DESCRIPTION OF THE PREFERRED EMBODIMENTS

The present invention is a modular bone anchor in the form of a pedicle screw for insertion into the vertebral body of a spinal vertebra via the pedicle. The bone anchor of the present invention may be inserted into a relatively small hole made through the cortical shell of the vertebra and into the vertebral body via the pedicle and thereafter compressed so as to expand in diameter for secure and selectively permanent engagement with the vertebra.

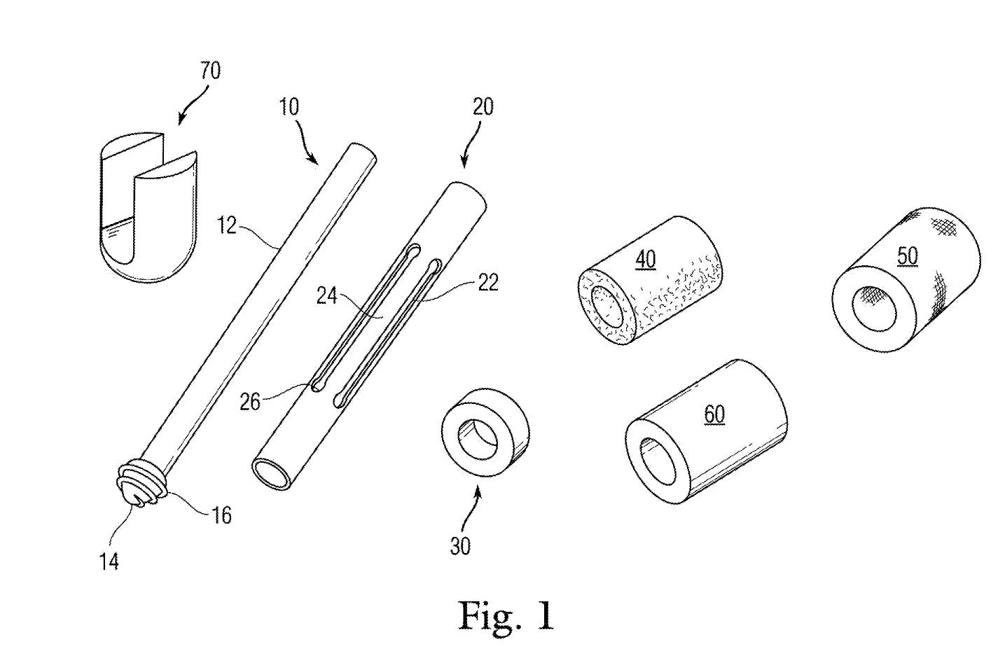

FIG. 1 is an exploded view of the elements of an embodiment of the invention prior to assembly and surgical insertion. Basic screw 10 is an elongate shaft preferably round in cross section, and from 30 mm to 60 mm long (up to 2½ inches). Basic screw 10 has a shank 12 of constant diameter substantially along its entire length all the way to one end.

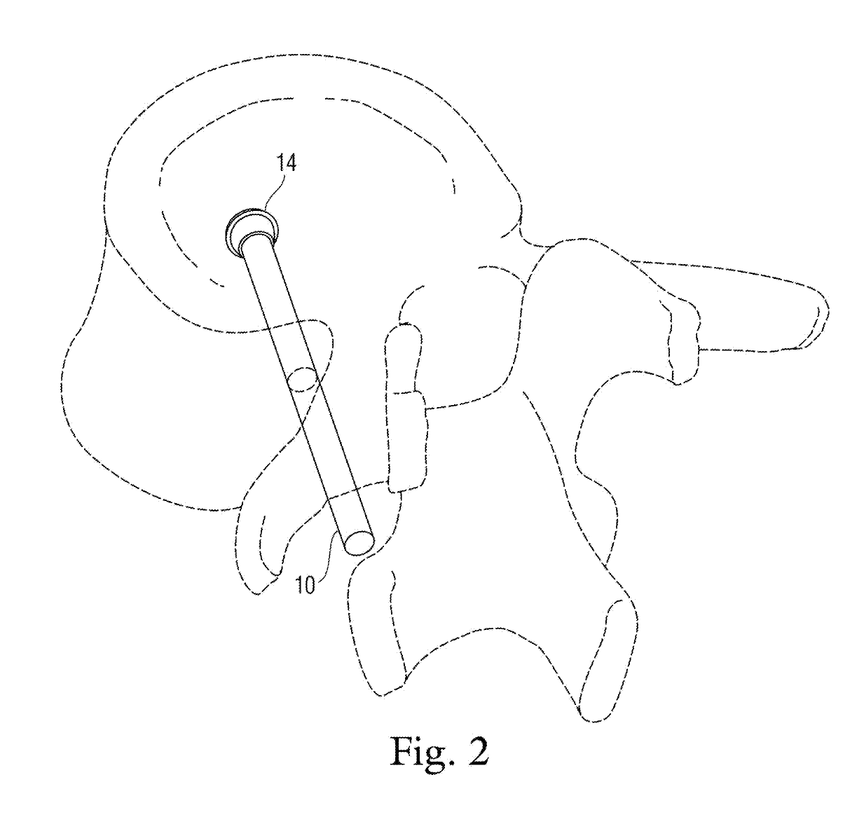

As seen in FIG. 2, the other end is provided with a proximal tip 14 for insertion into a predrilled hole through the cortical bone of the vertebral pedicle and into the cancellous bone of the vertebral body. Proximal tip 14 is an enlarged bulbous tip having a medial diameter greater than that of shank 12 and tapering down to a rounded point at its distal end. The exterior surface of the proximal tip 14 is provided with threads 16 for engagement with the interior surface of the hole prepared in the bone. The hole diameter is equal to the maximum root (minor) diameter of the tapered, threaded proximal end. Basic screw 10 is preferably made of titanium or other biocompatible materials such as stainless steel or PEEK.

Sleeve 20 is tubular in form and preferably cylindrical, having an inside diameter sufficient to for free insertion over the smaller end of the shank 12 of basic screw 10, leaving little or no play between the sleeve 20 and the shank 12. The outside diameter of sleeve 20 is less than or equal to the root diameter of the proximal tip 14 of the basic screw 10. Sleeve 20 is provided with an annular arrangement of longitudinal slots 22 centered approximately midway along its length and extending along a major portion of the length, although slots 22 may be shifted in either direction with respect to the longitudinal center. In a preferred embodiment, six slots 22 are equally spaced at 60 degree intervals about the longitudinal axis, and the slots define six bendable ribs 24 there between. One skilled in the art will appreciate that a greater or lesser number of slots 22/ribs 24 may be used with a corresponding adjustment of the equal angular spacing. The longitudinal slots 22 do not extend the entire length and therefore leave two annular collars 19 at each end of sleeve 20. When the collars 19 are compressed together with sufficient bias the ribs 24 bow outward to create a folding star 25.

In certain embodiments the physiology of a particular patient may best be accommodated by an unequal spacing of the longitudinal slots such that the ultimate pattern of the ribs 24 of the folding star 25 are not evenly distributed about the central axis.

Referring back to FIG. 1, longitudinal slots 22 may, in some embodiments, be as narrow as a laser-cut slit but are preferably approximately 5 to 15 degrees such that the ribs 24 formed between adjacent slits are approximately 45-55 degrees each. Slots 22 are each provided at both ends with a generally circular enlargement 26. Enlargement 26 is several degrees wider (in diameter) than the slot 22 itself and serves to create a slight necking or narrowing in width of the ribs 24 where the ribs join the un-slotted collars 19 at either end of the sleeve 20. The necking of the material at these points controls deformation of the ribs under compressive load during implantation and creation of the star pattern during surgery, as further described below. Sleeve 20 is preferably formed of a plastically deformable biocompatible metal or thermoplastic having similar characteristics.

A compression ring 30 is loaded onto the basic screw 10 behind the sleeve 20. The compression ring 20 is tubular and preferably cylindrical having an inside diameter sufficient to securely receive the shank of the basic screw 10 with little or no play between the sleeve and the shank. The compression ring 20 has an outside diameter greater than or equal to the outside diameter of the sleeve 20 but not greater than the root diameter of the distal tip 14. Compression ring 30 is preferably formed of a biocompatible metal such as titanium or stainless steel but may be made of polymers having sufficient strength and hardness.

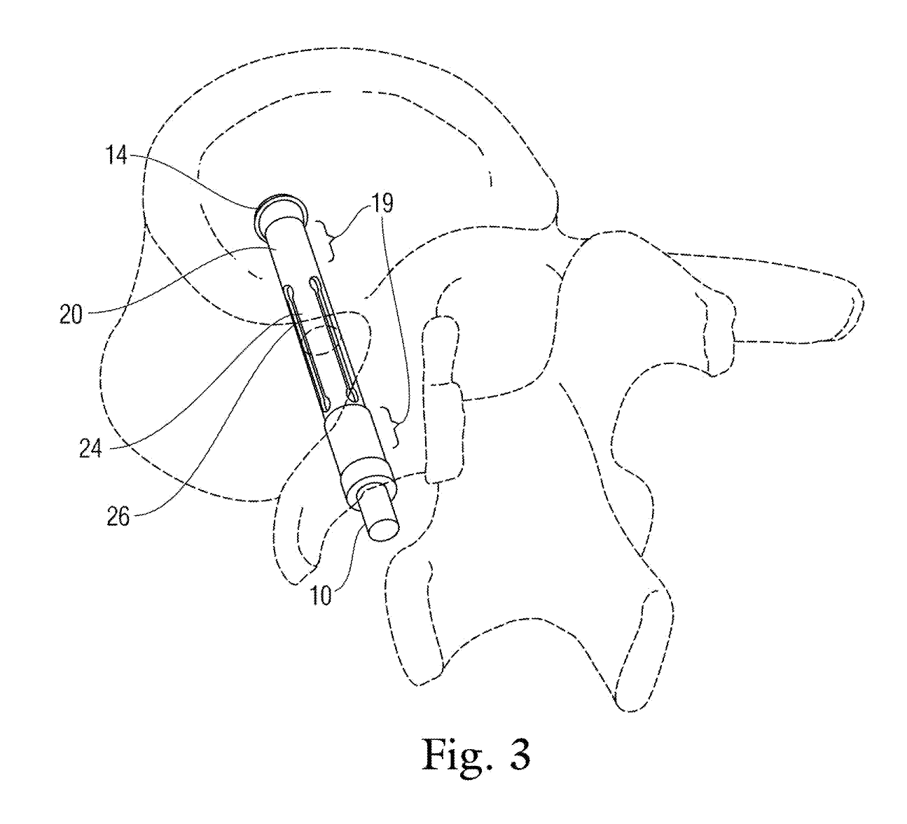

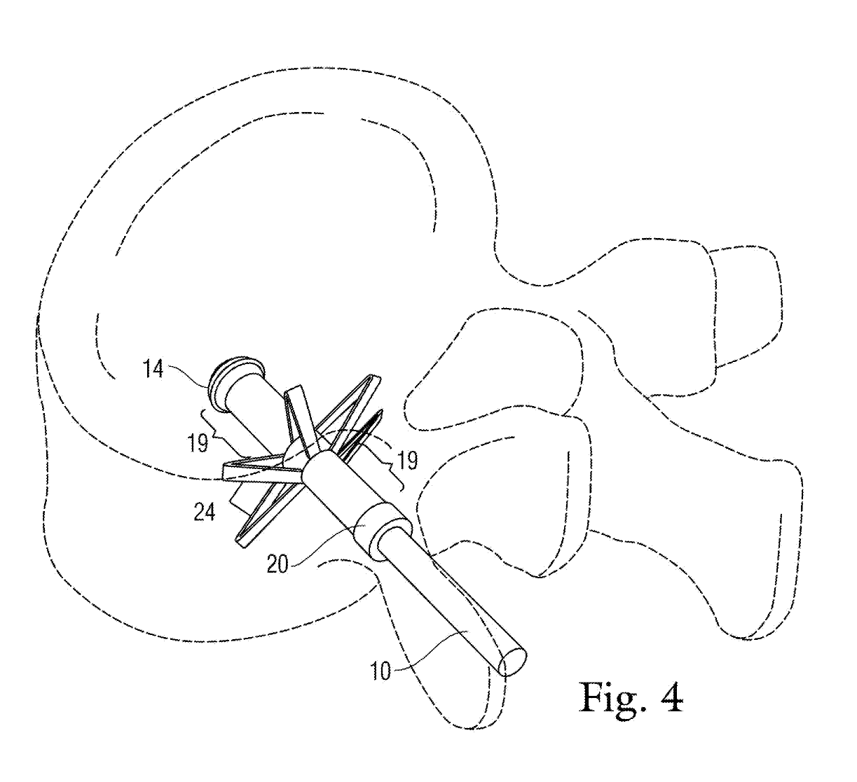

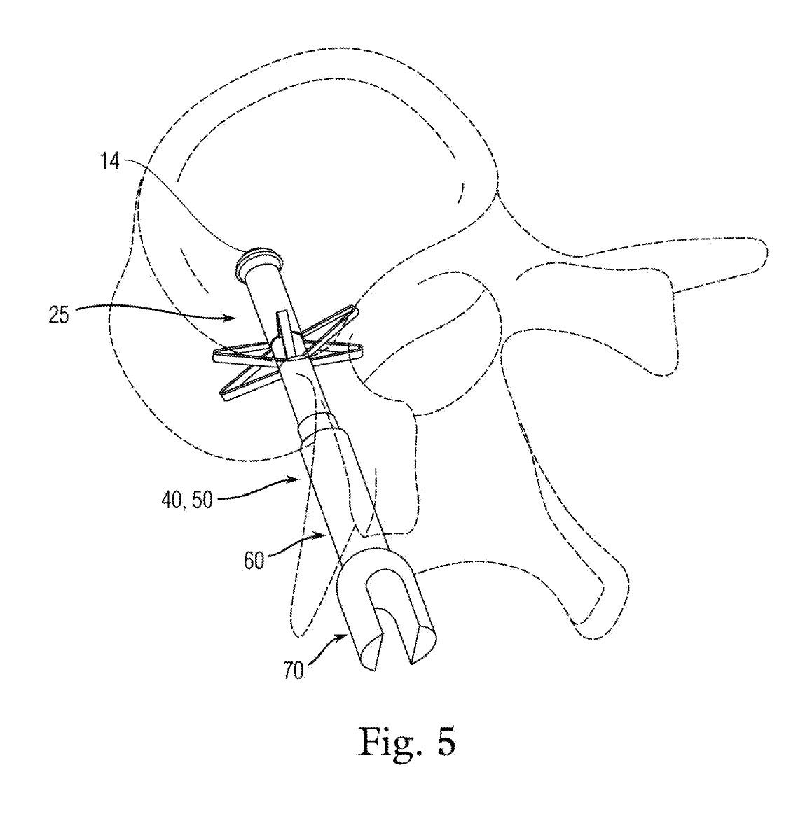

With combined reference to FIGS. 2 through 5, during surgery a posterior approach to the vertebrae to be stabilized is executed and a hole prepare in the bone to receive the basic screw 10. The hole is prepared using a conventional surgical drill and enters the bone at the lamina and extends through the pedicle and into the vertebral body. The diameter of the hole is selected by the surgeon to be approximately equal to the root diameter of the anticipated basic screw 10 and in conjunction with the anticipated orthopedic instrumentation and the physiology of the patient. Basic screw 10 is rotated into the hole such that threads 16 engage the bone surface and draw the basic screw into the hole. The distal end of the basic screw 10 may be provided with opposing flat surfaces so as to permit the screw 10 to be better gripped and rotated by a tool. In an alternate embodiment the distal end of basic screw 10 may be provided with a keyed recess such as a hexagonal (non-round) recess or a transverse slot for engagement with a tool to turn the screw. The basic screw 10 is preferably headless.

After the basic screw 10 has been positioned in the hole the surgeon selects a sleeve 20 and slips the sleeve 20 over the distal end of the screw sliding it down the shank until the inserted end of the sleeve contacts the proximal tip 14. The sleeve length, the length of the end collar 19 inserted into the hole and the length slots 22/ribs 24 are selected in conjunction with the depth of the hole such that the longitudinal midpoint of the ribs 24 is positioned at or just inside the inner surface of the hard cortical bone shell of the vertebral body when the inserted end of the sleeve 20 contacts the proximal tip 14. Compression collar 30 is slipped over the distal end of the basic screw 10 and advanced to contact the adjacent end of the sleeve 20.

Force is applied to the compression collar 30 urging it toward the proximal tip 14 of the basic screw. Advancement of the compression collar 30 toward the proximal tip 14 compresses the sleeve 20, reducing its overall length and thereby deforming the ribs 24 which are forced to deform outward and away from the longitudinal axis by the presence of the shank of the basic screw 10. The necking at the ends of the ribs 24 produces a weak point controlling deformation such that each rib 24 similarly bends at the weak points where the ribs join the collars. Rib 24 also bends at its midpoint (the point of greatest moment) such that the ribs are folded generally in half and extend radially outward in a folding star 25 form. Folding star 25 exceeds the diameter of the original hole and resists withdrawal by engaging the cortical bone shell and distributing any applied loads over a greater area.

Shank 12 is preferably smooth walled. Alternatively, shank 12 may be provided with one or more rows of teeth along the length of the outside surface. A plier-like leveraging tool (not shown) may have one jaw positioned over the distal end of the shank, and another engaged with the teeth, so as to grip the shank and drive the compression collar 30 toward the proximal tip. A leveraging tool may also engage the outer surface of the vertebra to simultaneously draw the basic screw 10 back out of the hole a small amount, advancing the proximal tip 14 toward the compression collar 30 and drawing the expanding folding star up against the inner surface of the cortical bone shell. A ratcheting or anti-reverse mechanism may be provided on the inner surface of compression ring 30 for cooperative engagement with the teeth, such that compression ring 30 is prevented from retreating along the shaft after the applied force is removed.

In an alternate embodiment shank 12 is formed with external thread for cooperative engagement with threads formed on the inner surface of compression ring 30. Compression ring 30 may be driven along the shank 12 toward distal tip 14 by rotation of the compression ring 30 by application of a pin wrench or similar tool. In yet another alternate embodiment the inner surface of the compression ring 30 is smooth walled and is driven toward the proximal tip by placing a non-round nut having a threaded inner surface and an outside diameter smaller than the diameter of the hole in the bone such that the nut can be rotates by application of convention socket to drive the compression ring 30 toward the proximal tip 14. The nut mayor may not be removed after use at the discretion of the surgeon.

After the basic screw 10 has been positioned in the vertebral hole and the star patter 25 expanded to secure it in place, a variety of adaptive elements may be position on the exposed distal end of the basic screw 10 for ancillary purposes such as bone grafting. In the illustrated embodiment, exemplary adaptive elements include a cylindrical tubular collar of sintered bone 40 (such as True Bone Ceramics), a textile 50 or other similar graft cage type material having an inside diameter sufficient to securely receive the distal end of the shank of the basic screw 10 and having an outside diameter less than or equal to the diameter of the hole prepared in the bone. Collars 40,50 (which may be utilized together or in the alternative) promote eventual bone growth around the shank at the cortical wall to further secure the implant in the long term. Other exemplary collars 40, 50 may include medicine or analgesic-leaching materials.

Next, a tubular elastomeric collar 60 is also inserted onto the distal end of the shank of the basic screw 10. The tubular elastomeric collar 60 has an inside diameter sufficient to securely receive the distal end of the shank of the basic screw 10 by friction. Elastomeric collar 60 also has an outside diameter less than or equal to the diameter of the hole prepared in the bone so as to be partially recessed within the bone hole.

Finally, a fixation element such as a tulip 70 may be attached distally for engagement with external pedicle screw spacers/fixation members. The illustrated fixation element 70 is a tulip-type rod seat 70 inserted onto the distal end of the shank of the basic screw 10 (outside the elastomeric collar 60) for engaging a pedicle screw connecting rod. The tulip-type rod seat 70 protrudes out of the hole prepared in the bone.

The above-described system provides a pedicel-screw-type bone anchor that is resistant to pull out failure. Moreover, since the present bone anchor does not rely entirely on the engagement of the threads of a screw with the exposed bone surfaces of a hole drilled in the vertebra of a patient for its strength and ability to remain fixed in place, it can be utilized in conjunction with bone that has previously been damaged by pullout failure of a bone screw. The “Kebab” configuration also allows for easy insertion and placement of a variety of other ancillary elements such as bone grafts and/or medicament-leaching graft promoters.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth herein.

What is claimed:

1. A bone anchor comprising
a screw having a shank portion and proximal portion, the proximal portion having a diameter greater than the shank portion, said proximal portion characterized by external threads for engagement with a hole prepared in a bone;
a tubular sleeve having a first end and a second end and sized for insertion over said shank portion of the said screw wherein said first end is engaged with said proximal portion, said sleeve having a plurality of longitudinal slots arranged about the longitudinal axis of said sleeve thereby forming a plurality of longitudinal ribs there between;
a tubular collar for insertion over said shank portion wherein said collar is engaged with said second end of said sleeve.

2. The bone anchor of claim 1 further comprising
a tubular collar of natural or synthetic bone growth promoting material for insertion over said shank portion wherein said bone growth promoting material collar is advanced down said shank so as to contact said bone;
an elastomeric collar for insertion over said shank portion, said elastomeric extending beyond the distal end of said shank portion; and
a rod seat for receiving an orthopedic rod affixed to said elastomeric collar.

3. A method of securing a bone anchor to a bone comprising the steps of:
providing a pre-drilled hole in a bone;
inserting into said hole a bone screw having a shank portion and proximal portion, the proximal portion having a diameter greater than the shank portion, said proximal portion characterized by external threads, said thread engaging the surface of said bone within said hole so as to advance said proximal portion of said screw into said hole on rotation of said shank portion;
inserting a tubular sleeve having a first end and a second end over said shank portion of the said screw and into said hole wherein said first end is engaged with said proximal portion of said bone screw, said sleeve having a plurality of longitudinal slots arranged about the longitudinal axis of said sleeve thereby forming a plurality of longitudinal ribs there between;
inserting a compression collar over said shank portion of the said screw and into said hole wherein said compression collar is engaged with said second end of said sleeve;
driving said compression collar down said shank toward said proximal end thereby compressing said sleeve and causing said ribs form a radial pattern of arms substantially perpendicular to said longitudinal axis, said pattern having a diameter greater than the diameter of said hole.

4. The method of claim 3 further comprising inserting over said shank portion a collar of natural or synthetic bone growth promoting material, said bone growth promoting material collar being advanced down said shank so as to contact said bone.

5. The method of claim 3 further comprising inserting over said shank portion an elastomeric collar, said elastomeric extending beyond the distal end of said shank portion.

6. The method of claim 5 further comprising inserting a rod seat on to said elastomeric collar for receiving an orthopedic rod.
